(19) United States
(12) Patent Application Publication
(10) Pub. No.: US 2016/0081912 A1
(43) Pub. Date: Mar. 24, 2016

(54) COMPOSITIONS FOR IMPROVING THE HAIR, SKIN, AND NAILS

(71) Applicant: FYBRANDS CORP., Salt Lake City, UT (US)

(72) Inventors: James Humphreys, Victoria (CA); Max Sabour, Salt Lake city, UT (US)

(21) Appl. No.: 14/891,290

(22) PCT Filed: May 15, 2014
(86) PCT No.: PCT/US14/38245

§ 371 Date: Nov. 13, 2015

Related U.S. Application Data
(60) Provisional application No. 61/823,779, filed on May 15, 2013.

(51) Int. Cl. A61K 8/97; A61Q 7/00; A61Q 5/00; A61Q 19/00; A61K 8/37; A61K 8/35

(52) U.S. Cl. CPC A61K 8/97; A61K 8/37; A61K 8/35; A61Q 5/00; A61Q 19/00; A61Q 7/00

(57) ABSTRACT

Compositions for improving the health and appearance of human hair, skin, and nails, and methods of using and preparing such compositions are disclosed.

### CROSS REFERENCE TO RELATED APPLICATIONS

This application claims priority to U.S. Provisional Patent Application No. 61/823,779, which was filed May 15, 2013 and entitled “COMPOSITIONS FOR STIMULATION OF HUMAN HAIR GROWTH AND METHOD OF PREPARATION,” which is incorporated by reference in its entirety.

### TECHNICAL FIELD

The present disclosure is directed to compositions for improving the health and appearance of human hair, skin, and nails, and methods of preparing and using such compositions.

### BACKGROUND

Most men and women want healthy, beautiful hair, skin, and nails. These tissues also serve important physiological functions. Skin, for example, forms a protective barrier against infection by pathogens. Unfortunately, there are many factors that can damage these tissues.

Hair may be damaged by chemicals in care and treatment products, UV light, heat, and mechanical stress from styling. Genetic factors also affect hair density. Moreover, hair density frequently decreases with age and may be affected by other factors such as stress.

Likewise, skin may be damaged by UV light, chemicals, the drying effects of wind, and certain infectious agents, including certain bacteria, viruses, and fungi. The health and appearance of skin also typically decrease with age.

It is therefore readily apparent that there is a great need for effective compositions to improve the health and appearance of human hair, skin, and nails.

### DETAILED DESCRIPTION

The present disclosure provides compositions to deliver an active ingredient, cycloastragalus, to human hair, skin, and/or nails. Cycloastragalus (or cycloastragenol) is isolated from the Astragalus herb, which has been used in Chinese medicine for centuries.

In certain embodiments, the disclosure provides compositions to deliver the active ingredient to the root bulb of the hair follicle. Cycloastragalus is not soluble in water. Creating a cream formulation could possibly lead to clogging of pores at the site of the hair follicle, which could negatively affect restoration of the follicle. In certain aspects, the present disclosure relates to delivery of cycloastragalus using oil-based formulations. Oil is absorbed into the stratum corneum and deeper layers of the skin where the root bulb cells are located.

Initial attempts at determining an appropriate oil carrier were done using either or in combination together C10-30 Cholesterol and Lanosterol esters. While these carriers were successful for solubilizing the cycloastragalus, it was decided against since there was a possibility of creating cholesterol plugs which would actually inhibit healthy follicles and therefore hair growth.

Attempts were made to dissolve cycloastragalus into water with very limited results. Further attempts were then made in to dissolve the cycloastragalus in oil carrier. These met with limited success.

Cycloastragalus is soluble in acetone. We evaluated the possibility that cycloastragalus could be first dissolved in acetone and then put into solution with an oil carrier. Since acetone evaporates to completion out of any open system, it could be removed leaving the cycloastragalus in solution. One should keep in mind that acetone is incredibly flammable, and methods for removing acetone should be chosen carefully.

Certain aspects of the disclosure relate to compositions for stimulation of human hair growth. In certain embodiments, the composition comprises an effective amount of cycloastragalus and an oil carrier. In some embodiments, the oil carrier comprises capric/capryllic triglycerides. Capric/capryllic acid triglycerides may be derived, for example, from glycerine and coconut oil or palm kernel oil.

In certain embodiments, the cycloastragalus is present in the final composition at about 1%. In certain other embodiments, the cycloastragalus is present in the final composition at less than 1%, between about 0.1% and 1%, about 0.1%, or less than 0.1%.

In certain embodiments, the composition may comprise an essential oil. Many essential oils have a long history of medical use. The composition may include an essential oil such as rosemary, thyme, cedarwood, chamomile, lavender, and/or geranium. Other essential oils may also be used.

In certain embodiments, the composition may comprise herbal extracts including, but not limited to, saw palmetto extract.

In certain embodiments, the composition may include other ingredients including, but not limited to, one or more salts to balance tonicity, stabilizers, cleansers, thickening agents, and fragrances.

Certain aspects of the disclosure relate to methods of preparing compositions for administering cycloastragalus to the root bulb of the hair follicle. The method may include the steps of dissolving a desired amount of cycloastragalus into acetone, adding capric/capryllic triglyceride, and removing the acetone. “Removing the acetone” does not necessarily mean that 100% of the acetone is removed from the mixture; in certain embodiments, a residual amount of acetone may remain in the composition. In certain embodiments, one or more essential oils, herbal extracts, or other ingredients may be added. In some embodiments, active oxygen species, such as ozone, may be used to thicken the composition to improve application.

As discussed above, the method of preparing the compositions may in some embodiments result in some acetone in the final composition. Without being bound by any particular theory, acetone may enhance the effects of the cycloastragalus in the composition by, e.g., enhancing absorption into the desired tissue.

Certain aspects of the disclosure relate to methods of improving hair density by administering a therapeutically effective amount of a composition comprising cycloastragalus to the scalp of a patient in need thereof. In certain embodiments, the administration may be on a periodic basis, such as daily, several times per week, or weekly.

Certain other aspects of the disclosure relate to methods of stimulating human hair growth using compositions comprising cycloastragalus.

The present disclosure also provides methods and compositions for improving the health and appearance of hair, skin, nails, and eyebrows. In certain embodiments, the disclosure provides a method for improving the health of at least one of hair, skin, nails, or eyebrows, comprising administering a composition which comprises an effective amount of cycloastragalus and capric/capryllic triglycerides. The composition may be applied to an area of hair, skin, nails, or eyebrows for which improved health is most desired. In certain other embodiments, the present disclosure provides a composition for improving the health of at least one of hair, skin, nails, or eyebrows, wherein said composition comprises an effective amount of cycloastragalus and capric/capryllic triglycerides. The composition may include any of the characteristics previously described.

Compositions for skin and/or nail care may be formulated as oil-based products, or they may be delivered as ointments, creams, or gels. Such compositions may be administered on a periodic basis, such as daily, several times per week, or weekly

To further illustrate these embodiments, the following examples are provided. These examples are not intended to limit the scope of the claimed invention, which should be determined solely on the basis of the attached claims.

### EXAMPLE 1

A total desired amount of cycloastragalus was dissolved into acetone at moderate temperature (40-50° C.). Capric/capryllic triglyceride was then added with mixing until completely mixed. The heat was then gently increased to evaporate the acetone completely (up to 60° C.). The solution was cooled with stirring. Rosemary EO 1% was added once the temperature was below 50° C., and the product was poured into dispensing containers for use by test subjects.

### EXAMPLE 2

A total amount of cycloastragalus was dissolved into acetone at low temperature (20-30° C.). Capric/capryllic triglyceride was then added with mixing until completely mixed. Using a rotovaporator, the acetone was completely removed (30 minutes maximum). Rosemary EO 1% was added with stirring.

An exemplary embodiment of the composition follows that would yield a 1% active following the evaporation of acetone:

| Compound | Amount | Percentage |
|---|---|---|
| Cycloastragalus | 1 | 1% |
| Capric/Capryllic | 98 | 82% |
| Triglycerides | | |
| Rosemary | 1 | 1% |
| Acetone | 20 | 17% |
| | 120 | 100% |

### EXAMPLE 3

Compositions comprising other cycloastragalus percentages (e.g., 0.1%) can be prepared using the methods recited in the previous examples.

### EXAMPLE 4

Compositions for improving the health and appearance of hair, skin, nails, and eyebrows can be prepared using the methods recited in the previous examples.

### EXAMPLE 5

Active oxygen species such as ozone may be injected into a composition by sparging with agitation. High-shear agitation may be used to create micelles.

It will be apparent to those having skill in the art that many changes may be made to the details of the above-described embodiments without departing from the underlying principles of the invention. The scope of the present invention should, therefore, be determined only by the following claims.

1-13. (canceled)

14. A composition for administering cycloastragalus to at least one of skin, nails, and eyebrows, said composition comprising:
an effective amount of cycloastragalus, and
an oil carrier.

15. The composition of claim 14, wherein the oil carrier comprises capric/capryllic triglycerides.

16-20. (canceled)

21. The composition of claim 14, further comprising an essential oil.

22. (canceled)

23. The composition of claim 14, further comprising an herbal extract.

24. The composition of claim 23, wherein the herbal extract is saw palmetto extract.

25. The composition of claim 14, further comprising a thickening agent.

26. The composition claim 14, further comprising one or more ingredients selected from the group consisting of salts, stabilizers, cleansers, thickening agents, and fragrances.

27-34. (canceled)

35. A method for improving the health of at least one of hair, skin, nails, or eyebrows, comprising administering a composition, said composition comprising an effective amount of cycloastragalus and an oil carrier.

36. The method of claim 35, wherein the oil carrier comprises capric/capryllic triglycerides.

37-42. (canceled)

43. A method for preparing a composition comprising cycloastragalus for improving the health of human hair, skin, or nails, said method comprising the steps of dissolving a desired amount of cycloastragalus into acetone, adding an oil carrier, and removing the acetone.

44. (canceled)

45. The method of claim 43, wherein the oil carrier comprises capric/capryllic triglycerides.

46. The method of any of claim 43, further comprising the step of adding an essential oil

47. The method of any of claim 43, further comprising the step of adding an herbal extract.

48. The method of any of claim 43, further comprising the step of adding one or more ingredients selected from the group consisting of salts, stabilizers, cleansers, thickening agents, and fragrances.

49. The method of claim 43, further comprising the step of adding an active oxygen species by sparging with agitation.

50. (canceled)

51. A method of improving dermatological health comprising administering on a periodic basis a therapeutically effective amount of a composition according to any of claim 14 to the skin of a patient in need thereof.

52. The method of claim 51, wherein the composition is administered daily.

53. The method of claim 51, wherein the composition is administered several times per week.

54. The method of claim 51, wherein the composition is administered weekly.

55. The composition of claim 14 further comprising acetone.

56-57. (canceled)
